(19) United States
(12) Patent Application Publication
(10) Pub. No.: US 2023/0056215 A1
(43) Pub. Date: Feb. 23, 2023

(54) Refrigerant Dispenser for Tick Removal

(71) Applicant: Joseph Peter Luongo, North Fort Myers, FL (US)

(72) Inventor: Joseph Peter Luongo, North Fort Myers, FL (US)

(21) Appl. No.: 17/408,609

(22) Filed: Aug. 23, 2021

(51) Int. Cl. A61B 17/50

(52) U.S. Cl. CPC A61B 17/50; A61B 2017/505

(57) ABSTRACT

A refrigerant dispenser for tick removal can include a main body, an actuator structure, and a cage. A main body can include a proximal end, a distal end, and a cavity that extends from the proximate end to the distal end, with the proximate end having an intake opening in communication with the cavity and being configured to receive a refrigerant canister therein. An actuator structure can be connected to the distal end and can be in communication with the cavity. A cage can include an upper portion connected to the actuator structure and in communication with the cavity, and a lower portion terminating at an exhaust opening. The lower portion can be configured to at least partially surround a tick, and the exhaust opening can be in communication with the cavity.

### FIELD OF THE INVENTION

The present invention relates to tick removal, and more specifically, to a refrigerant dispenser for tick removal.

### BACKGROUND OF THE INVENTION

Prior solutions have used refrigerants released from canisters to freeze or significantly cool ticks to assist with their subsequent removal.

### SUMMARY OF THE INVENTION

It is an object of the present invention to provide a refrigerant dispenser for tick removal.

It is another object of the present invention to provide a refrigerant dispenser for tick removal that includes functional utility.

The present invention provides a refrigerant dispenser that accepts a canister containing pressurized refrigerant and facilitates the dispensing of such refrigerant onto a tick, which can allow for safe removal of the tick from a host.

In an exemplary embodiment of the present invention, a refrigerant dispenser can include a main body, an actuator structure, and a cage.

In an exemplary aspect, such a main body can include a proximal end, a distal end, and a cavity that extends from the proximate end to the distal end, with the proximate end having an intake opening in communication with the cavity and being configured to receive a refrigerant canister therein.

In another exemplary aspect, such an actuator structure can be connected to the distal end and can also be in communication with the cavity.

In a further exemplary aspect, such a cage can include an upper portion connected to the actuator structure and can also be in communication with the cavity, while the lower portion can terminate at an exhaust opening.

In still another exemplary aspect, the lower portion can be configured to at least partially surround a tick, and the exhaust opening can be in communication with the cavity.

Accordingly, in operation, a refrigerant canister positioned within the cavity can be effectuated by application of downward force thereupon, with dispensed refrigerant traveling to and out of the exhaust opening.

In another exemplary aspect, such a main body can include at least one finger grip, which can be positioned towards the proximal end.

In a further exemplary aspect, such an actuator structure can be configured to receive a canister nozzle.

In additional exemplary aspects, such an actuator structure can be tapered and/or include an abutment.

In still another exemplary aspect, such a cage can include at least one vent and/or a plurality of prongs.

In a further exemplary aspect, the at least one vent can be configured to exhaust refrigerant at any desired angle, which can include an angle towards the proximal end.

In yet another exemplary aspect, the exhaust opening can be defined at least in part by a ring structure.

### BRIEF DESCRIPTION OF THE DRAWINGS

FIG. 1 illustrates an exemplary embodiment of a refrigerant dispenser having a main body, an actuator structure, and a cage.

FIG. 2 illustrates a side perspective of such an embodiment.

FIG. 3 illustrates a section view of such an embodiment.

FIG. 4 illustrates an exemplary refrigerant canister.

FIG. 5A illustrates a section view of an exemplary actuator structure and cage, with the actuator structure being tapered.

FIG. 5B illustrates a section view of such an exemplary actuator structure and cage dispensing refrigerant.

FIG. 6A illustrates a section view of an exemplary actuator structure and cage, with the actuator structure having an abutment.

FIG. 6B illustrates a section view of such an exemplary actuator structure and cage dispensing refrigerant.

FIG. 7 illustrates an exemplary manipulation of an exemplary refrigerant dispenser.

### DETAILED DESCRIPTION

An object of the present invention is to provide a refrigerant dispenser for tick removal.

It should be noted that this disclosure includes a plurality of embodiments each having a plurality of elements, aspects, and/or steps and such elements, aspects, and/or steps need not necessarily be interpreted as being conjunctively required by one or more embodiments of the present invention. In particular, all combinations of such elements, aspects, and/or steps can enable a separate embodiment of the present invention, which may be claimed with particularity in any future filed Patent Application. Moreover, such elements, aspects, and/or steps disclosed herein, whether expressly or implicitly, are to be construed strictly as illustrative and enabling, and not necessarily limiting. Therefore, it is expressly set forth that any elements, aspects, and/or steps, independently or in any combination of one of more thereof are merely illustratively representative of one or more embodiments of the present invention and are not to be construed as necessary in a strict sense.

Further, to the extent the same element, aspect, or step is defined differently anywhere within this disclosure, whether expressly or implicitly, the broader definition is to take absolute precedence, with the distinctions encompassed by the narrower definition to be strictly construed as optional.

Illustratively, perceived benefits of the present invention can include functional utility, whether expressly or implicitly stated herein, or apparent herefrom. However, it is expressly set forth that these benefits are not intended as exclusive. Therefore, any explicit, implicit, or apparent benefit from the disclosure herein is expressly deemed as applicable to the present invention.

According to the present invention, a refrigerant dispenser can be formed from any one or more materials or combinations of materials, such as one or more of plastic, rubber, wood, metal, crystalline material, or any other man-made or naturally occurring material, for example and not in limitation, insofar as functionally consistent with the present invention. Further, such a device can be manufactured in any one or more functionally compatible manners, such as through molding, cutting, machining, etc.

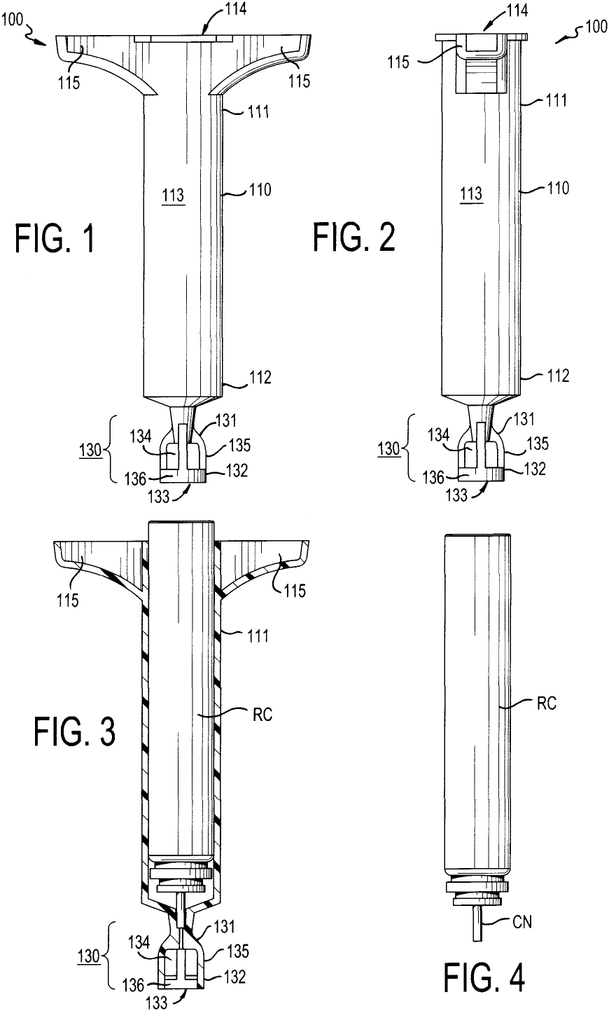

FIGS. 1-3 illustrate an exemplary embodiment of the present invention, in which a refrigerant dispenser 100 can include a main body 110, an actuator structure 120, and a cage 130. FIG. 4 illustrates an exemplary refrigerant canister RC containing a refrigerant under pressure, and can include a canister nozzle CN that can open a valve and release such refrigerant when moved towards canister RC.

In an exemplary aspect of the present invention, main body 110 can include a proximal end 111, a distal end 112, and a cavity 113 that extends from the proximate end to the distal end. Notably, main body 110 can be provided with any functionally compatible shape or shapes, and dimensions. Likewise, cavity 113 can be provided with any desired shape and dimensions configured to receive refrigerant canister RC. Further, refrigerant canister RC can include any functionally compatible refrigerant desired.

In another exemplary aspect, proximate end 111 can include an intake opening 114 in communication with cavity 113 and can be configured to receive a refrigerant canister RC therein as illustrated in FIG. 3.

In yet another exemplary aspect, actuator structure 120 can be connected to distal end 112 and can be in communication with cavity 113. In operation, which is further described below, actuator structure 120 can facilitate actuation of a refrigerant canister so as to release a refrigerant therefrom.

In still another exemplary aspect, cage 130 can include an upper portion 131 and a lower portion 132. Upper portion 131 can be connected to actuator structure 120 and can be in communication with cavity 113; whilst lower portion 132 can terminate at an exhaust opening 133.

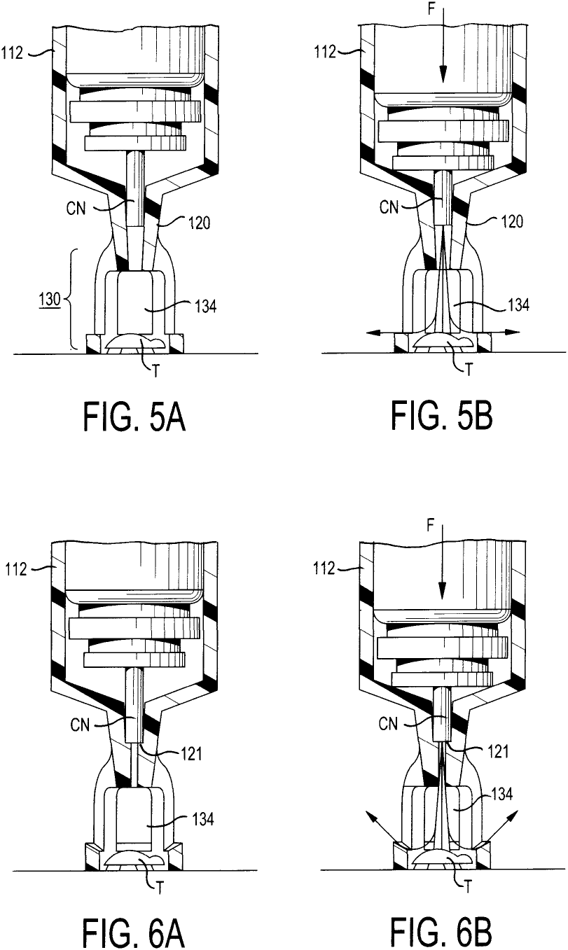

In still yet another exemplary aspect, lower portion 132 can be configured to at least partially surround a tick (as illustrated in FIGS. 5A-6B), and exhaust opening 133 can be in communication with cavity 113. Accordingly, refrigerant dispensed from canister RC can pass from cavity 113 and/or actuator structure 120 and reach exhaust opening 133, so as to freeze or otherwise cool a captured tick.

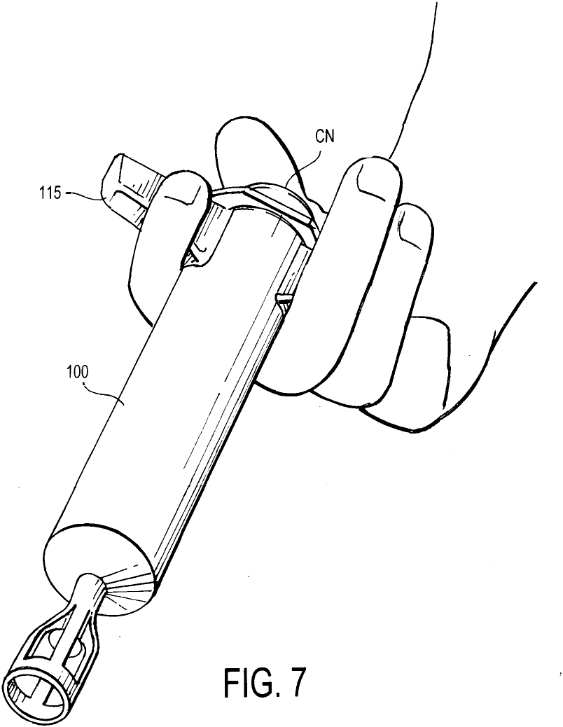

As further illustrated in FIG. 7, main body 110 can optionally include at least one finger grip 115 to facilitate the holding of dispenser 100 and actuation of canister nozzle CN. While finger grip 115 is illustratively shown being positioned towards proximal end 111 (which can include at proximal end 111), the same can be positioned at any functional position along main body 110.

As additionally illustrated in FIGS. 5A-6B, actuator structure 120 can be configured to receive canister nozzle therein.

In one exemplary embodiment, as illustrated in FIGS. 5A and 5B, actuator structure 120 can be tapered so as to partially receive canister nozzle CN before acting as an abutment preventing the same from moving further therethrough, which can provide actuation of the canister nozzle to release a refrigerant from refrigerant canister RC when a downward force F is applied to the refrigerant canister.

In another exemplary embodiment, as illustrated in FIGS. 6A and 6B, actuator structure 120 can include an abutment 121 or shelf for preventing the same from moving further therethrough, which can provide actuation of the canister nozzle to release a refrigerant from refrigerant canister RC when downward force F is applied to the refrigerant canister.

As further illustrated in FIGS. 5A-6B, cage 130 can include at least one vent 134 to allow excess refrigerant to flow in any desired direction other than directly towards the skin of a host, which can reduce the risk of skin burns to the host. As illustrated in FIG. 5B, vent 134 can be configured to allow excess refrigerant to flow is a direction perpendicular to main body 110; whereas as illustrated in FIG. 6B, such a vent can be configured to allow excess refrigerant to flow in a direction toward proximal end 111 or away from tick T.

As additionally illustrated in FIGS. 5A-6B, exhaust opening 133 can be defined at least in part by a ring structure 136. Notably, prongs 135 and/or ring structure 135 can function to trap tick T for subsequent freezing.

It will be apparent to one of ordinary skill in the art that the manner of making and using the claimed invention has been adequately disclosed in the above-written and attached description of the exemplary embodiments and aspects of the present invention.

It should be understood, however, that the invention is not necessarily limited to the specific embodiments, aspects, arrangement, and components shown and described above, but may be susceptible to numerous variations within the scope of the invention.

Therefore, the specification and drawings are to be regarded in an illustrative and enabling, rather than a restrictive, sense.

Accordingly, it will be understood that the above description of the embodiments of the present invention are susceptible to various modifications, changes, and adaptations, and the same are intended to be comprehended within the meaning and range of equivalents apparent to one of ordinary skill in the art.

Therefore I claim:

1. A refrigerant dispenser for tick removal, comprising:
a main body having a proximal end, a distal end, and a cavity that extends from the proximate end to the distal end, the proximate end having an intake opening in communication with the cavity and being configured to receive a refrigerant canister therein;
an actuator structure connected to the distal end and in communication with the cavity; and
a cage having an upper portion connected to said actuator structure and in communication with the cavity, and a lower portion terminating at an exhaust opening;
wherein the lower portion is configured to at least partially surround a tick, and the exhaust opening is in communication with the cavity.

2. The dispenser of claim 1, wherein said main body includes at least one finger grip.

3. The dispenser of claim 2, wherein the at least one finger grip is positioned towards the proximal end.

4. The dispenser of claim 1, wherein said actuator structure is configured to receive a canister nozzle.

5. The dispenser of claim 4, wherein said actuator structure is tapered.

6. The dispenser of claim 4, wherein said actuator includes an abutment.

7. The dispenser of claim 1, wherein said cage includes at least one vent.

8. The dispenser of claim 7, wherein said cage includes a plurality of prongs.

9. The dispenser of claim 7, wherein said at least one vent is angled towards the proximal end.

10. The dispenser of claim 1, wherein the exhaust opening is defined at least in part by a ring structure.
